(19) United States
(12) Patent Application Publication
(10) Pub. No.: US 2014/0074109 A1
(43) Pub. Date: Mar. 13, 2014

(54) SYSTEM AND METHOD FOR REMOVING AN OBJECT FROM WITHIN A MAMMAL

(71) Applicant: Northwestern University, Evanston, IL (US)

(72) Inventor: Scott A. Resnick, Chicago, IL (US)

(73) Assignee: Northwestern University, Evanston, IL (US)

(21) Appl. No.: 14/023,333

(22) Filed: Sep. 10, 2013

Related U.S. Application Data
(60) Provisional application No. 61/698,874, filed on Sep. 10, 2012.

(51) Int. Cl. A61B 17/50

(52) U.S. Cl. CPC A61B 17/50
USPC 606/113

(57) ABSTRACT

An object-removal system for removing an object from within a mammal includes a first tubular sheath, a second tubular sheath, a beveled cutting-blade, and a snare. The second tubular sheath is disposed coaxially inside the first tubular sheath. The beveled cutting-blade is attached to a distal end of the second tubular sheath. The snare is disposed within the second tubular sheath for attaching to an object.

### CROSS-REFERENCE TO RELATED APPLICATIONS

This application claims the benefit of priority to U.S. provisional patent application Ser. No. 61/698,874 filed Sep. 10, 2012, and entitled “SYSTEM AND METHOD FOR REMOVING AN OBJECT FROM WITHIN A MAMMAL,” the contents of which are herein incorporated by reference in their entirety.

### FIELD OF THE DISCLOSURE

This disclosure relates to a system and method for removing an object from within a mammal.

### BACKGROUND

It is often desired to remove objects from within a mammal As an example, for various reasons it may be desired to remove a filter which was initially inserted into the inferior vena cava of a mammal to prevent deep vein thrombus from migrating to the lung and causing a potentially life-threatening pulmonary embolism. However due to tissue of the inferior vena cava attaching to the filter after insertion, it is often difficult to remove the filter from the inferior vena cava. Sometimes these filters are left in place due to this removal difficulty. Some current systems and methods utilize lasers to cut the tissue to allow the filter to be removed from the inferior vena cava. However, the use of lasers is expensive and can lead to further complications if misapplied.

A system and method for removing objects from within a mammal is needed to overcome one or more issues associated with one or more of the current systems and methods.

### SUMMARY

In one embodiment, an object-removal system for removing an object from within a mammal includes a first tubular sheath, a second tubular sheath, a beveled cutting-blade, and a snare. The second tubular sheath is disposed coaxially inside the first tubular sheath. The beveled cutting-blade is attached to a distal end of the second tubular sheath. The snare is disposed within the second tubular sheath for attaching to an object.

In another embodiment, an object-removal system for removing an object from within a mammal includes a first tubular sheath, a second tubular sheath, and a snare. The second tubular sheath is disposed coaxially inside the first tubular sheath. A distal end of the second tubular sheath includes a beveled cutting-edge having a sharpness in a range of 0.1 N to 10.0 N. The snare is disposed within the second tubular sheath is for attaching to an object.

In still another embodiment, a method is disclosed of removing an object from within a mammal In one step, an object-removal system is inserted within a mammal The object-removal system includes a first tubular sheath, a second tubular sheath, and a snare. The second tubular sheath is disposed coaxially inside the first tubular sheath. A distal end of the second tubular sheath includes a beveled cutting-edge. The snare is disposed within the second tubular sheath. In another step, the snare is attached to an object within the mammal In an additional step, the second tubular sheath is moved in a longitudinal direction along the co-axis of the first and second tubular sheaths to force the beveled cutting-edge against tissue of the mammal attached to the object to cut the tissue with the beveled cutting-edge, as the second tubular sheath moves in the longitudinal direction, so that the object can be removed from the mammal In yet another step, the object-removal system and the object are removed from the mammal.

The scope of the present disclosure is defined solely by the appended claims and is not affected by the statements within this summary.

### BRIEF DESCRIPTION OF THE DRAWINGS

The disclosure can be better understood with reference to the following drawings and description. The components in the figures are not necessarily to scale, emphasis instead being placed upon illustrating the principles of the disclosure.

FIG. 1 illustrates a disassembled perspective view of one embodiment of an object-removal system;

FIG. 1A illustrates a disassembled perspective view of another embodiment of an object-removal system;

FIG. 2 illustrates an assembled perspective view of the object-removal system of FIG. 1;

FIG. 3 illustrates a cross-section view through line 3-3 of the object-removal system of FIG. 2;

FIG. 4 illustrates a perspective view of the object-removal system of FIGS. 1-3 inserted within an inferior vena cava of a mammal with a second tubular sheath, including a beveled cutting-edge, and a snare completely retracted within a hollow interior of a first tubular sheath;

FIG. 5 illustrates a perspective view of the object-removal system of FIG. 4 inserted within the inferior vena cava of the mammal with the snare extended from both the hollow interior of the first tubular sheath and from the hollow interior of the second tubular sheath with the snare attached to the object;

FIG. 6 illustrates a perspective view of the object-removal system of FIG. 5 inserted within the inferior vena cava of the mammal with the snare extended from the hollow interior of the first tubular sheath with the snare attached to the object, and with the second tubular sheath extended from the hollow interior of the first tubular sheath with the beveled cutting-edge disposed against the tissue cutting the tissue so that the object can be removed from the inferior vena cava;

FIG. 7 illustrates a perspective view of the object-removal system of FIG. 6 inserted within the inferior vena cava of the mammal with the snare extended from both the hollow interior of the first tubular sheath and from the hollow interior of the second tubular sheath with the snare attached to the object, and with the second tubular sheath and the beveled cutting-edge completely retracted back into the hollow interior of the first tubular sheath after the tissue has been cut with the beveled cutting-edge preventing the beveled cutting-edge from doing further cutting;

FIG. 8 illustrates a perspective view of the object-removal system of FIG. 7 inserted within the inferior vena cava of the mammal with the snare and the attached object completely retracted into both the hollow interior of the first tubular sheath and the hollow interior of the second tubular sheath, and with the second tubular sheath and the beveled cutting-edge also completely retracted in the hollow interior of the first tubular sheath;

FIG. 9 illustrates a perspective view of the object-removal system of FIG. 8 with the object-removal system and the attached object removed from the mammal;

FIG. 10 illustrates a perspective view of the object-removal system of FIG. 6 inserted within the inferior vena cava of the mammal with the snare extended from the hollow interior of the first tubular sheath and attached to the object, and with the second tubular sheath further extended from the hollow interior of the first tubular sheath so that the snare and the attached object are disposed completely within the hollow interior of the second tubular sheath after the tissue has been cut by the beveled cutting-edge of the second tubular sheath;

FIG. 11 illustrates a perspective view of FIG. 10 with the second tubular sheath removed from the inferior vena cava and from the mammal itself, thereby also removing the snare and the attached object from the inferior vena cava and from the mammal itself, with the first tubular sheath still disposed within the inferior vena cava of the mammal;

FIG. 12 illustrates a perspective view of FIG. 11 with the first tubular sheath removed from the inferior vena cava of the mammal;

FIG. 13 illustrates a perspective view of the object-removal system of FIG. 6 inserted within the inferior vena cava of the mammal with the first tubular sheath having been extended over the snare, over the attached object, and over the second tubular sheath so that each is completely disposed within the hollow interior of the first tubular sheath;

FIG. 14 illustrates a perspective view of FIG. 13 with the first tubular sheath removed from the inferior vena cava and from the mammal itself thereby also removing the snare, the attached object, and the second tubular sheath from the inferior vena cava and from the mammal itself; and

FIG. 15 is a flowchart illustrating one embodiment of a method of removing an object from within a mammal.

### DETAILED DESCRIPTION

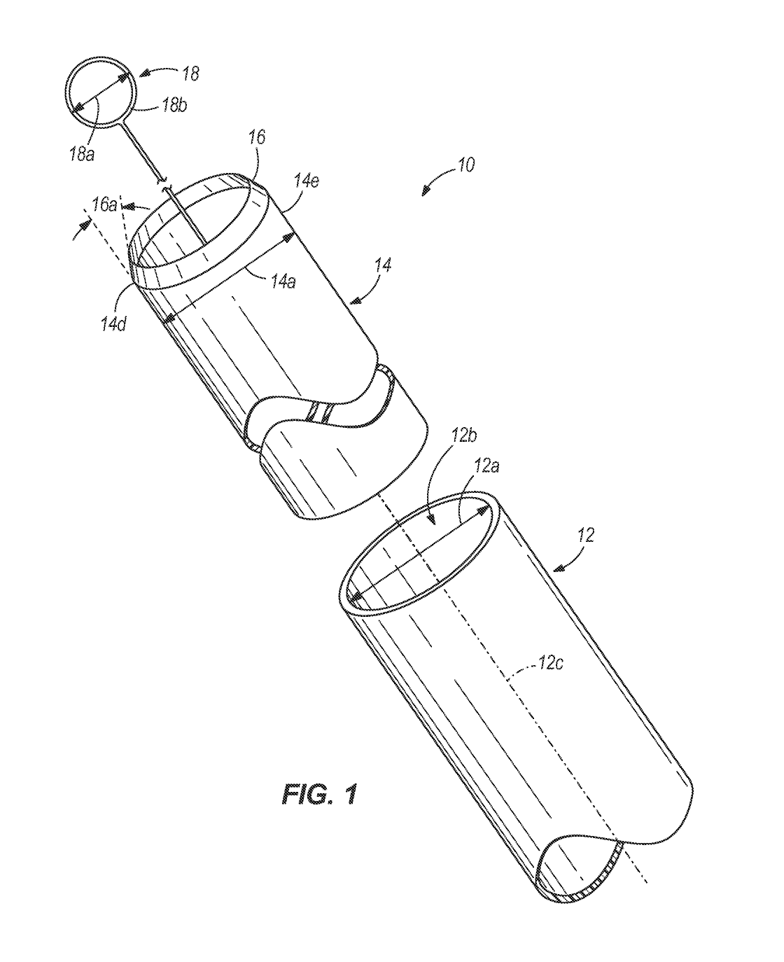

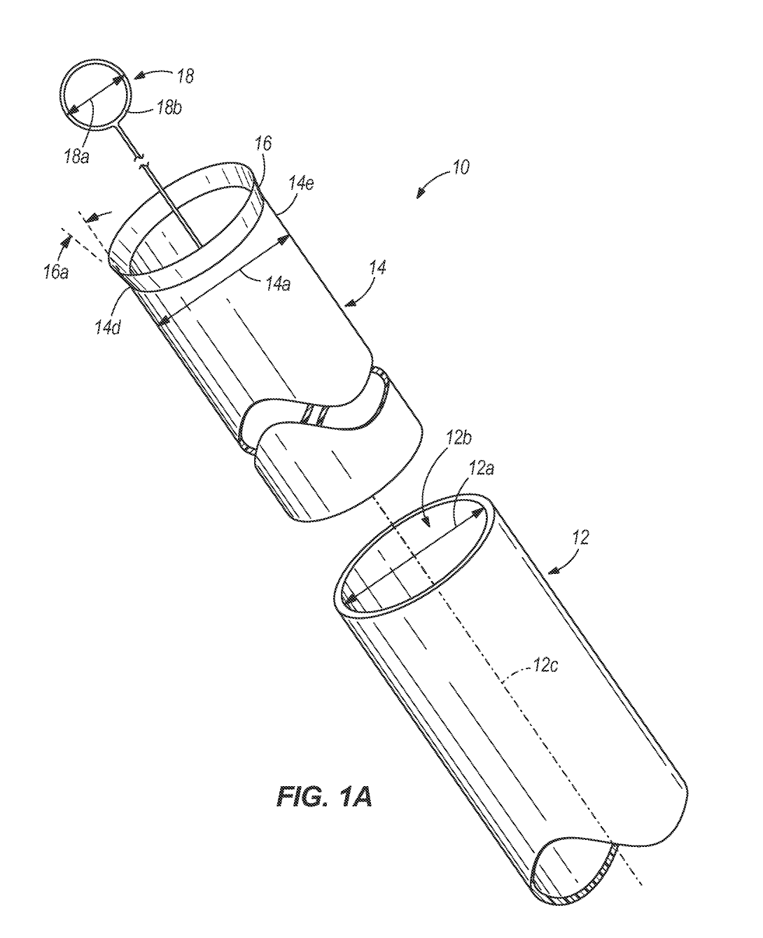

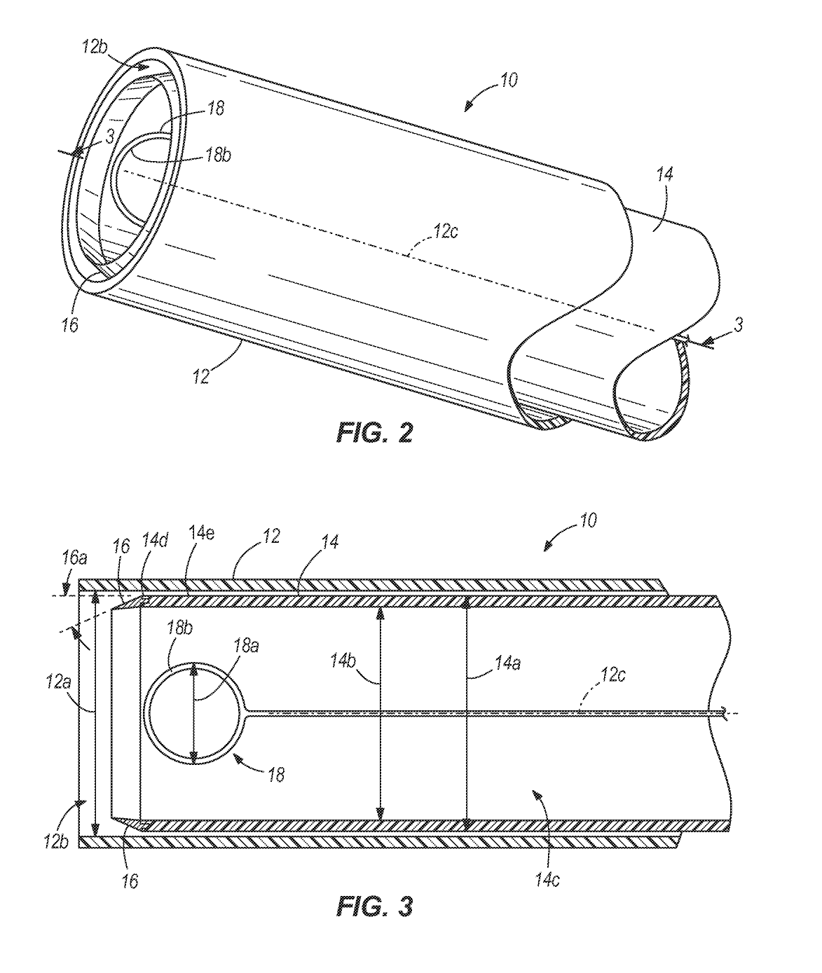

FIG. 1 illustrates a disassembled perspective view of one embodiment of an object-removal system 10. FIG. 2 illustrates an assembled perspective view of the object-removal system 10 of FIG. 1. FIG. 3 illustrates a cross-section view through line 3-3 of the object-removal system 10 of FIG. 2. As shown collectively in FIGS. 1-3, the object-removal system 10 comprises a first tubular sheath 12, a second tubular sheath 14, a beveled cutting-edge 16, and a snare 18. The first tubular sheath 12 has an inner diameter 12a which is larger than an outer diameter 14a of the second tubular sheath 14 and also larger than a width 18a of the snare 18 allowing the second tubular sheath 14 and the snare 18 to be completely retracted within, and extended from, a hollow interior 12b of the first tubular sheath 12. When the snare 18 and the second tubular sheath 14 are disposed within the first tubular sheath 12, they are disposed coaxially relative to axis 12c of the first tubular sheath 12. The second tubular sheath 14 has an inner diameter 14b which is larger than the width 18a of the snare 18 allowing the snare 18 to be completely retracted within, and extended from, a hollow interior 14c of the second tubular sheath 14.

A distal end 14d of the second tubular sheath 14 comprises the beveled cutting-edge 16 with the beveled-cutting edge 16 attached to the distal end 14d. The beveled cutting-edge 16 may be attached to the distal end 14d of the second tubular sheath 14 using varying attachment means such as molding, snap-fits, fasteners, or through other types of attachment means. The first tubular sheath 12 is made of a flexible material, such as a flexible plastic having a wire winding, having stiffness in a range of 0.01 to 100.0 pounds per inch. The second tubular sheath 14 is made of a flexible material, such as a flexible plastic having a wire winding, having stiffness in a range of 0.01 to 100.0 pounds per inch. The beveled-cutting edge 16 comprises a sharp cutting-blade. The beveled cutting-edge 16 has a sharpness in a range of 0.1 N to 10.0 N. The beveled cutting-edge 16 is made of metal such as steel or other material which can be cut, sharpened, or constructed to form a cutting blade edge. The beveled cutting-edge 16 is symmetrically disposed around a perimeter of the distal end 14d of the second tubular sheath 14. The beveled cutting-edge 16 is disposed inwardly at an angle 16a relative to an outer surface 14e of the second tubular sheath 14 in a range of 5 to 85 degrees. The snare 18 comprises a loop 18b. In other embodiments, the snare 18 may comprise a varying number of loops. The snare 18 is made of a single or multiple wire loops which may be made of a wide variety of materials. In other embodiments, the first tubular sheath 12, the second tubular sheath 14, the beveled cutting-edge 16, and the snare 18 may be made of varying materials, may comprise varying shapes and sizes, may vary in number, and may be disposed in varying configurations relative to one another.

FIG. 1A illustrates a disassembled perspective view of another embodiment of the object-removal system 10. As shown in FIG. 1A, the beveled cutting-edge 16 may be disposed outwardly at an angle 16a relative to the outer surface 14e of the second tubular sheath 14 in a range of 5 to 85 degrees. In still other embodiments, the beveled cutting-edge 16 may vary in orientation, configuration, location, size, number, or material.

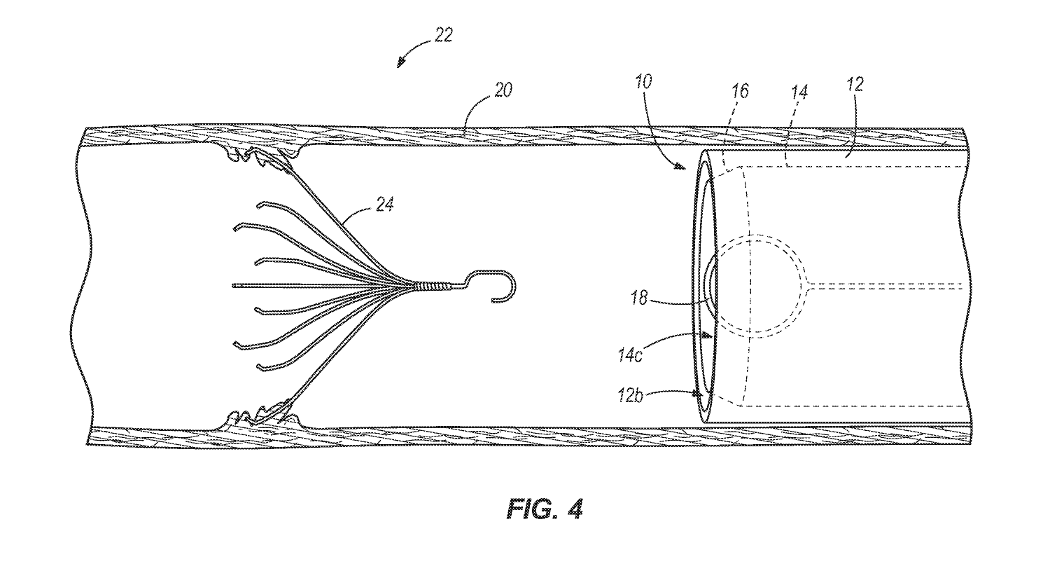

FIGS. 4-9 illustrate chronological steps that may be followed to remove an object 24 from a mammal 22. FIG. 4 illustrates a perspective view of the object-removal system 10 of FIGS. 1-3 inserted within an inferior vena cava 20 of a mammal 22 with the second tubular sheath 14, including the beveled cutting-edge 16, and the snare 18 completely retracted within the hollow interior 12b of the first tubular sheath 12. The snare 18 is also completely retracted within the hollow interior 14c of the second tubular sheath 14. The object-removal system 10 may be used to remove the object 24 from within the inferior vena cava 20 of the mammal 22. The object 24 may comprise a filter, such as an Inferior Vena Cava filter or other type of filter, or another type of object. In other embodiments, the object-removal system 10 may be used to remove an object 24 from varying portions of the mammal 22.

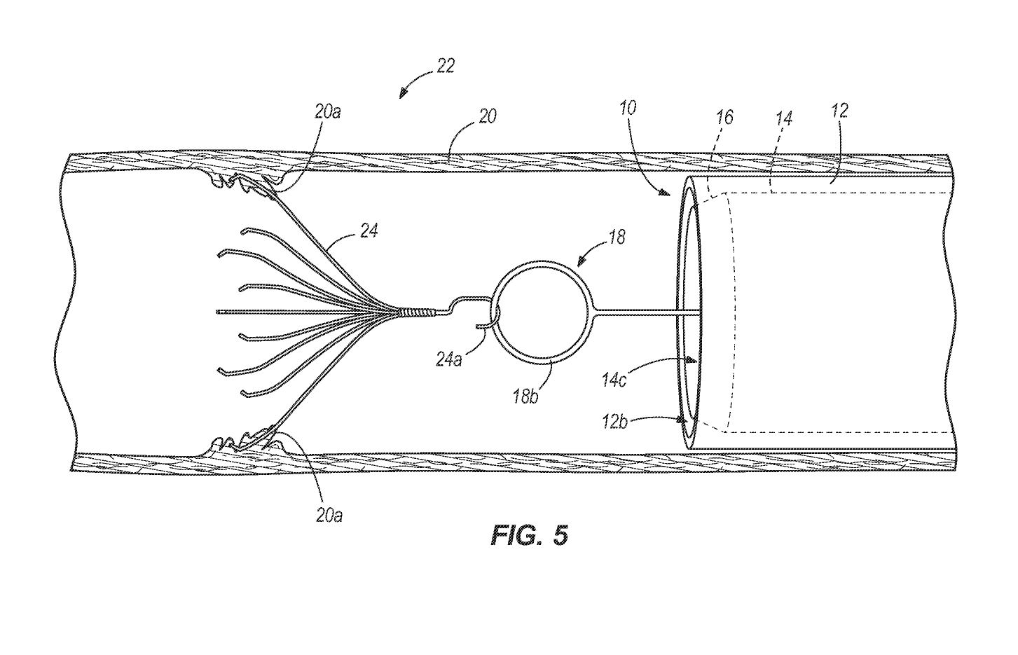

FIG. 5 illustrates a perspective view of the object-removal system 10 of FIG. 4 inserted within the inferior vena cava 20 of the mammal 22 with the snare 18 extended from both the hollow interior 12b of the first tubular sheath 12 and from the hollow interior 14c of the second tubular sheath 14 with the snare 18 attached to the object 24. The snare 18 may be attached to the object by hooking a hook 24a of the object 24 into the loop 18b of the snare 18. The second tubular sheath 14, including the beveled cutting-edge 16, is completely retracted within the hollow interior 12b of the first tubular sheath 12. Tissue 20a of the inferior vena cava 20 is attached to the object 24 preventing the snare 18 from removing the object 24 from the inferior vena cava 20.

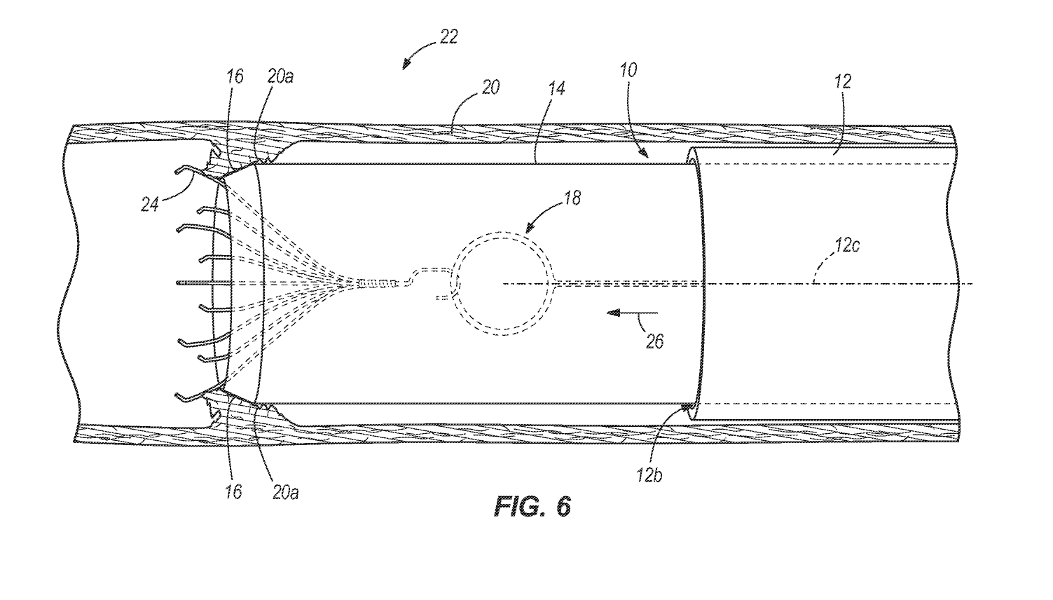

FIG. 6 illustrates a perspective view of the object-removal system 10 of FIG. 5 inserted within the inferior vena cava 20 of the mammal 22 with the snare 18 extended from the hollow interior 12b of the first tubular sheath 12 with the snare 18 attached to the object 24, and with the second tubular sheath 14 extended from the hollow interior 12b of the first tubular sheath 12 with the beveled cutting-edge 16 disposed against the tissue 20a cutting the tissue 20a so that the object 24 can be removed from the inferior vena cava 20. During this cutting action, the second tubular sheath 14 is moved in a longitudinal direction 26 along the co-axis 12c of the first tubular sheath 12, the second tubular sheath 14, and the snare 18. It is noted that due to the sharpness of the beveled cutting-edge 16, no rotation of the beveled cutting-edge is required to cut the tissue 20a so that the object 24 can be removed from the inferior vena cava 20.

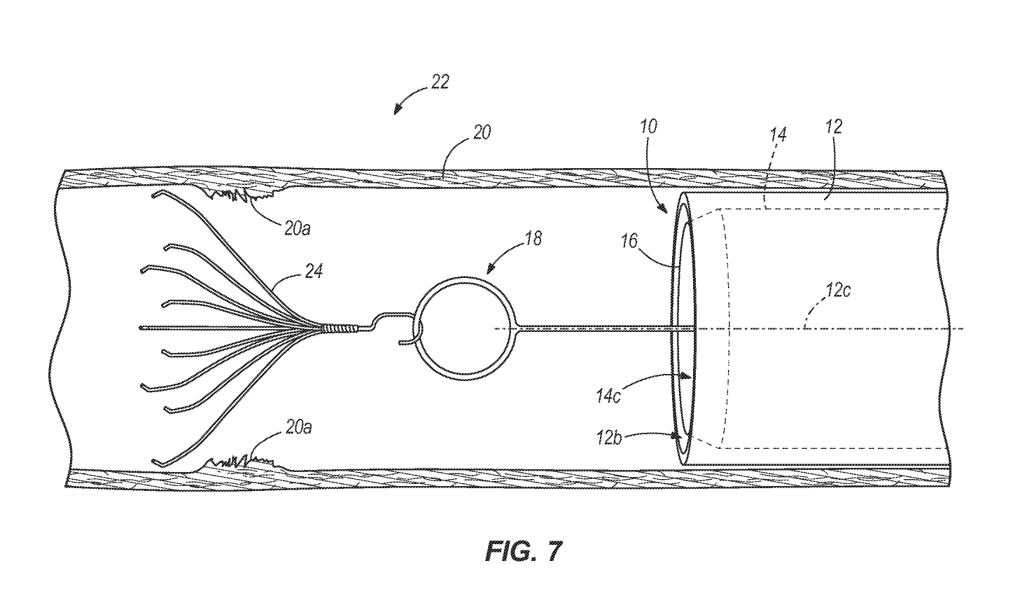

FIG. 7 illustrates a perspective view of the object-removal system 10 of FIG. 6 inserted within the inferior vena cava 20 of the mammal 22 with the snare 18 extended from both the hollow interior 12b of the first tubular sheath 12 and from the hollow interior 14c of the second tubular sheath 14 with the snare 18 attached to the object 24, and with the second tubular sheath 14 and the beveled cutting-edge 16 completely retracted back into the hollow interior 12b of the first tubular sheath 12 after the tissue 20a has been cut with the beveled cutting-edge 16 preventing the beveled cutting-edge 16 from doing further cutting.

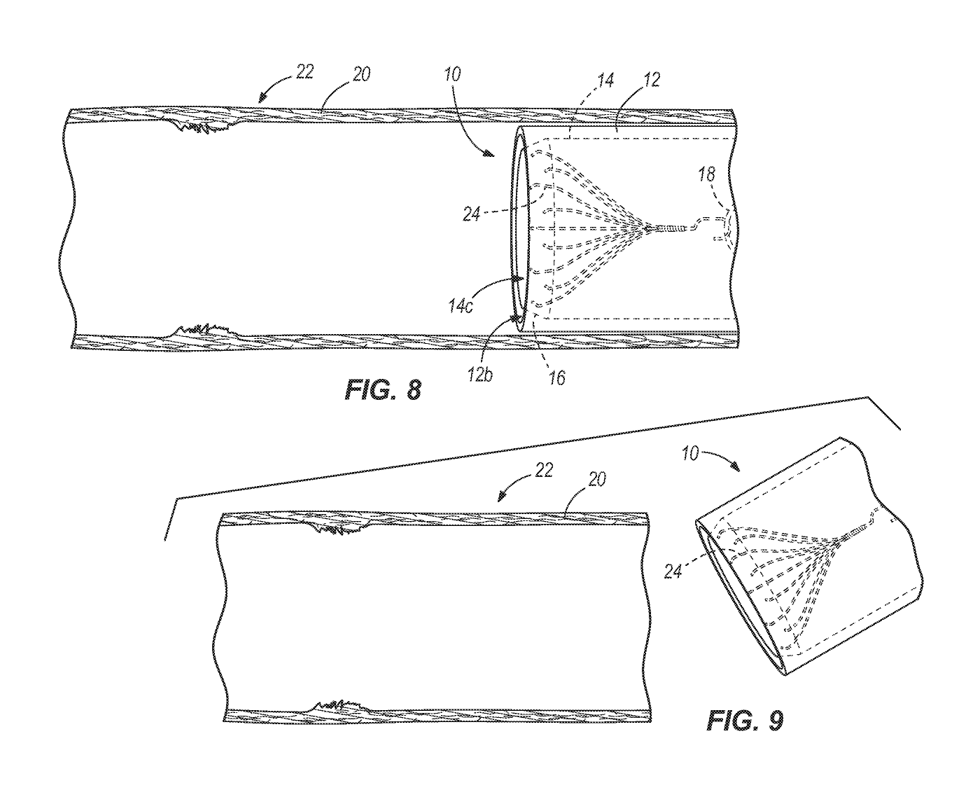

FIG. 8 illustrates a perspective view of the object-removal system 10 of FIG. 7 inserted within the inferior vena cava 20 of the mammal 22 with the snare 18 and the attached object 24 completely retracted into both the hollow interior 12b of the first tubular sheath 12 and the hollow interior 14c of the second tubular sheath 14. The second tubular sheath 14 and the beveled cutting-edge 16 are also completely retracted in the hollow interior 12b of the first tubular sheath 12.

FIG. 9 illustrates a perspective view of the object-removal system 10 of FIG. 8 with the object-removal system 10 and the attached object 24 removed from the inferior vena cava 20 and from the mammal 22 itself.

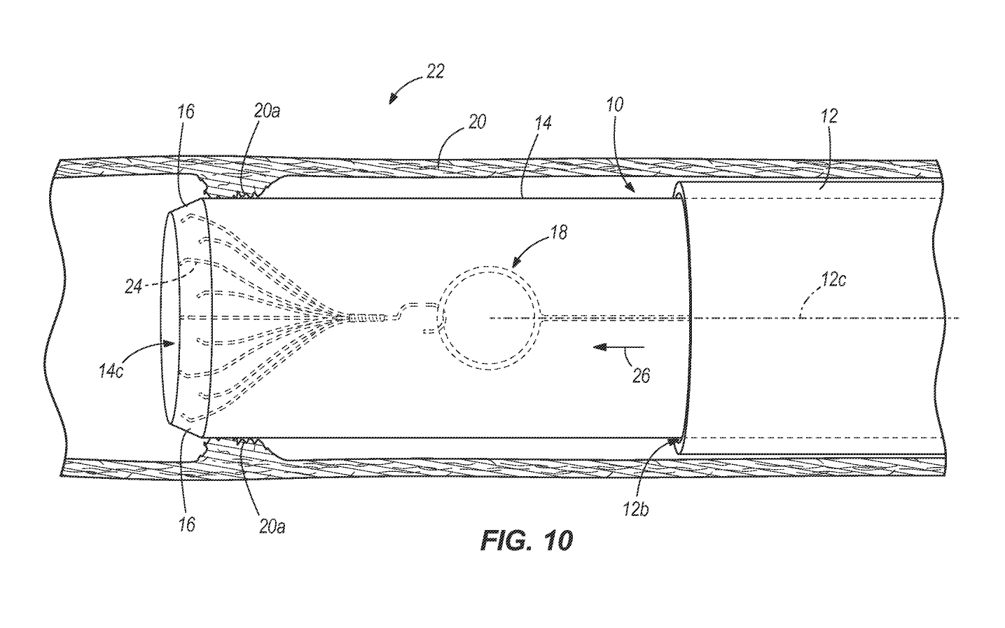

In an alternative embodiment, instead of following the chronological steps of FIGS. 7-9 after the step of FIG. 6, the chronological steps of FIGS. 10-12 may be followed. FIG. 10 illustrates a perspective view of the object-removal system 10 of FIG. 6 inserted within the inferior vena cava 20 of the mammal 22 with the snare 18 extended from the hollow interior 12b of the first tubular sheath 12 and attached to the object 24, and with the second tubular sheath 14 further extended from the hollow interior 12b of the first tubular sheath 12 so that the snare 18 and the attached object 24 are disposed completely within the hollow interior 14c of the second tubular sheath 14 after the tissue 20a has been cut by the beveled cutting-edge 16 of the second tubular sheath 14.

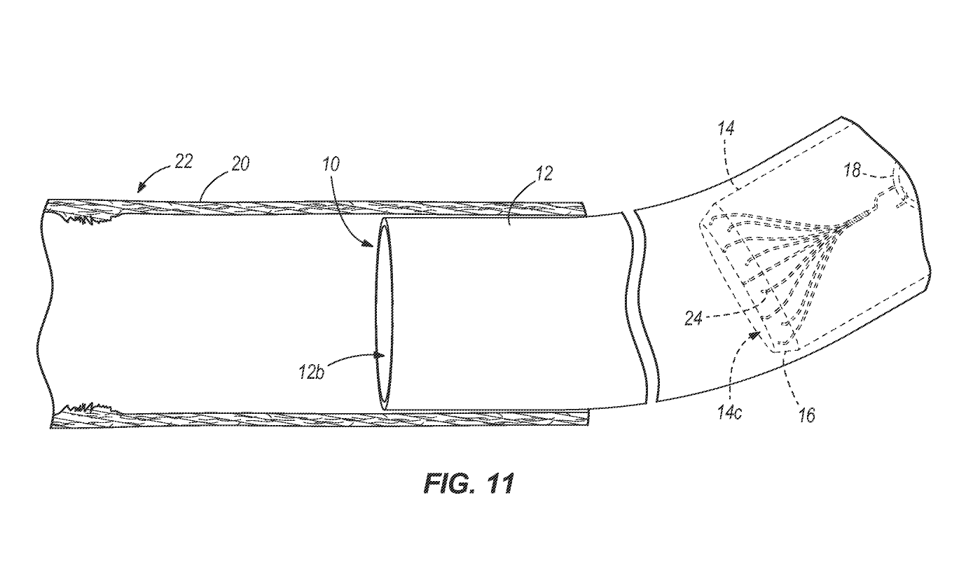

FIG. 11 illustrates a perspective view of FIG. 10 with the second tubular sheath 14 removed from the inferior vena cava 20 and from the mammal 22 itself, thereby also removing the snare 18 and the attached object 24 from the inferior vena cava 20 and from the mammal 22 itself, with the first tubular sheath 12 still disposed within the inferior vena cava 20 of the mammal 22.

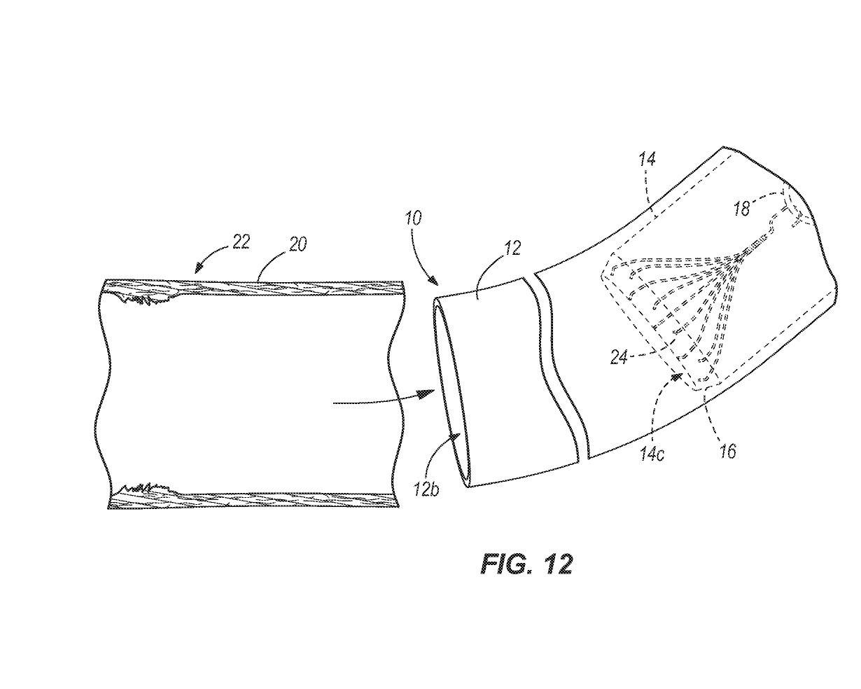

FIG. 12 illustrates a perspective view of FIG. 11 with the first tubular sheath 12 removed from the inferior vena cava 20 of the mammal 22.

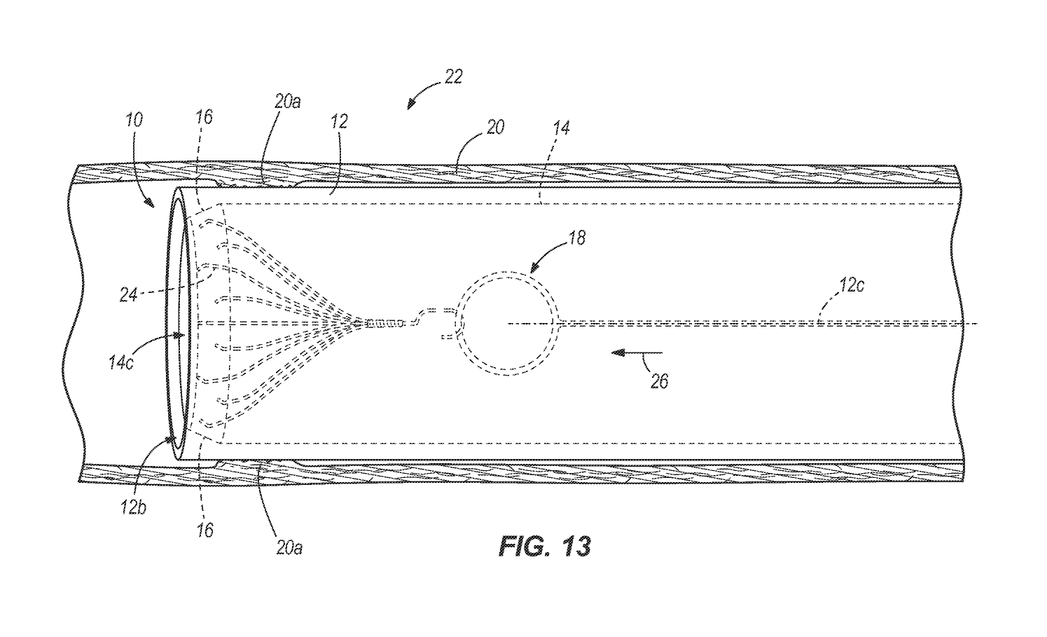

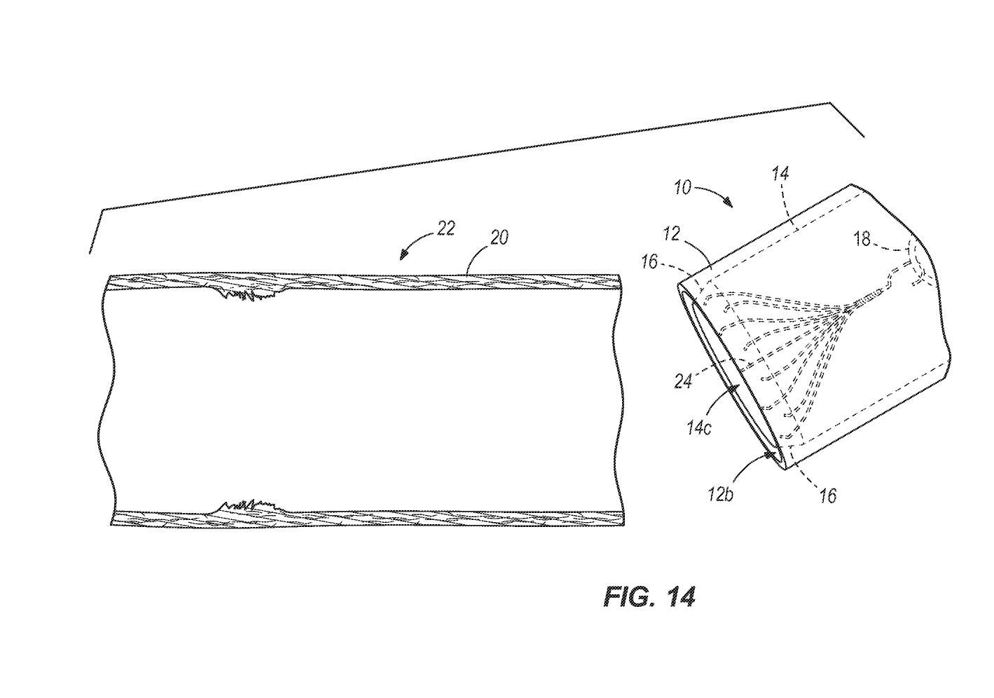

In yet another alternative embodiment, instead of following the chronological steps of FIGS. 7-9 after the step of FIG. 6, the chronological steps of FIGS. 13-14 may be followed. FIG. 13 illustrates a perspective view of the object-removal system 10 of FIG. 6 inserted within the inferior vena cava 20 of the mammal 22 with the first tubular sheath 12 having been extended over the snare 18, over the attached object 24, and over the second tubular sheath 14 so that each is completely disposed within the hollow interior 12b of the first tubular sheath 12.

FIG. 14 illustrates a perspective view of FIG. 13 with the first tubular sheath 12 removed from the inferior vena cava 20 and from the mammal 22 itself thereby also removing the snare 18, the attached object 24, and the second tubular sheath 14 from the inferior vena cava 20 and from the mammal 22 itself.

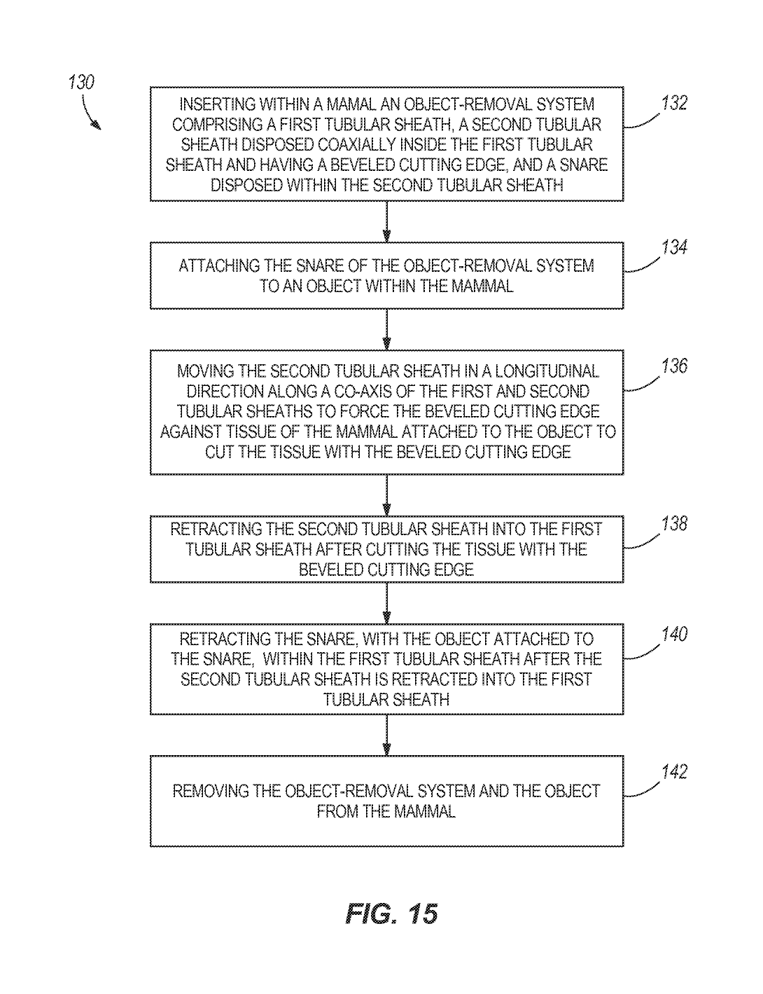

FIG. 15 is a flowchart illustrating one embodiment of a method 130 of removing an object from within a mammal The object-removal system 10 of FIGS. 1-3 or FIG. 1A may be used during implementation of the method 130. In step 132, an object-removal system is inserted within a mammal The object-removal system may be inserted within an inferior vena cava of the mammal The object-removal system may comprise a first tubular sheath, a second tubular sheath disposed coaxially inside the first tubular sheath, and a snare disposed within the second tubular sheath. A distal end of the second tubular sheath may comprise a beveled cutting-edge. The beveled cutting-edge may be made of metal such as steel or other material which can be cut, sharpened, or constructed to form a cutting blade edge. The beveled cutting-edge may have a sharpness in a range of 0.1 N to 10.0 N. The beveled cutting-edge may comprise a blade attached to the distal end of the second tubular sheath. The beveled cutting-edge may be symmetrically disposed around a perimeter of the distal end of the second tubular sheath. The first tubular sheath may comprise a flexible material, such as a flexible plastic having a wire winding, having stiffness in a range of 0.01 to 100.0 pounds per inch. The second tubular sheath may comprise a flexible material, such as a flexible plastic having a wire winding, having stiffness in a range of 0.01 to 100.0 pounds per inch. The snare may comprise a single loop or multiple loops.

In step 134, the snare of the object-removal system is attached to an object within the mammal In one embodiment, step 134 may further comprise extending the snare from the first tubular sheath to attach the snare to the object while the second tubular sheath is disposed within the first tubular sheath. The object may comprise a filter disposed within an inferior vena cava of the mammal The snare may be attached to the object by engaging a hook of the object onto a loop of the snare. In step 136, the second tubular sheath is moved in a longitudinal direction along the co-axis of the first and second tubular sheaths to force the beveled cutting-edge against tissue of the mammal attached to the object to cut the tissue with the beveled cutting edge, as the second tubular sheath moves in the longitudinal direction, so that the object can be removed from the mammal In one embodiment, step 136 may further comprise extending the second tubular sheath from the first tubular sheath after the snare is attached to the object within the mammal In step 138, the second tubular sheath is retracted into the first tubular sheath after cutting the tissue with the beveled cutting-edge. In step 140, the snare is retracted, with the object attached to the snare, within the first tubular sheath after the second tubular sheath is retracted into the first tubular sheath. In step 142, the object-removal system and the object are removed from the mammal.

In other embodiments of the method 130, one or more steps of the method may be modified in substance or order, not followed, or one or more additional steps may be followed. For instance, in one alternative embodiment instead of following steps 138-142, the following substitute steps may be followed: the second tubular sheath may be further extended from the hollow interior of the first tubular sheath so that the snare and the attached object are disposed completely within the hollow interior of the second tubular sheath after the tissue has been cut by the beveled cutting-edge of the second tubular sheath; the second tubular sheath may be removed from the inferior vena cava and from the mammal itself, thereby also removing the snare and the attached object from the inferior vena cava and from the mammal itself, with the first tubular sheath still disposed within the inferior vena cava of the mammal; and the first tubular sheath may be removed from the inferior vena cava of the mammal.

In another alternative embodiment instead of following steps 138-142, the following substitute steps may be followed: the first tubular sheath may be extended over the snare, over the attached object, and over the second tubular sheath so that each is completely disposed within the hollow interior of the first tubular sheath; and the first tubular sheath may be removed from the inferior vena cava and from the mammal itself thereby also removing the snare, the attached object, and the second tubular sheath from the inferior vena cava and from the mammal itself.

One or more embodiments of the disclosure may reduce one or more issues experienced by one or more current systems or methods for removing objects from within a mammal by providing an easy-to-use, simple, inexpensive system and method, with minimal chance of complications, for removing objects from within a mammal.

The Abstract is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the disclosure is defined by the appended claims. Accordingly, the disclosure is not to be restricted except in light of the appended claims and their equivalents.

1. An object-removal system for removing an object from within a mammal comprising:
a first tubular sheath;
a second tubular sheath disposed coaxially inside the first tubular sheath;
a beveled cutting-blade attached to a distal end of the second tubular sheath; and
a snare disposed within the second tubular sheath for attaching to an object.

2. The object-removal system of claim 1 wherein the first tubular sheath is made of a flexible material having a stiffness in a range of 0.01 to 100.0 pounds per inch.

3. The object-removal system of claim 1 wherein the second tubular sheath is made of a flexible material having a stiffness in a range of 0.01 to 100.0 pounds per inch.

4. The object-removal system of claim 1 wherein the beveled cutting-blade has a sharpness in a range of 0.1 N to 10.0 N.

5. The object-removal system of claim 1 wherein the beveled cutting-blade is made of metal, or other material shaped into a cutting blade edge.

6. The object-removal system of claim 5 wherein the second tubular sheath is made of plastic, with or without supportive wire winding or other supportive element.

7. The object-removal system of claim 1 wherein the beveled-cutting blade is symmetrically disposed around a perimeter of the distal end of the second tubular sheath.

8. The object-removal system of claim 1 wherein the snare comprises a loop or loops.

9. An object-removal system for removing an object from within a mammal comprising:
a first tubular sheath;
a second tubular sheath disposed coaxially inside the first tubular sheath, a distal end of the second tubular sheath comprising a beveled cutting-edge having a sharpness in a range of 0.1 N to 10.0 N; and
a snare disposed within the second tubular sheath for attaching to an object.

10. The object-removal system of claim 9 wherein the beveled cutting-edge comprises a blade attached to the distal end of the second tubular sheath.

11. The object-removal system of claim 9 wherein the first tubular sheath is made of a flexible material having a stiffness in a range of 0.01 to 100.0 pounds per inch.

12. The object-removal system of claim 9 wherein the second tubular sheath is made of a flexible material having a stiffness in a range of 0.01 to 100.0 pounds per inch.

13. The object-removal system of claim 9 wherein the beveled cutting-blade is made of metal or other material shaped into a cutting blade edge.

14. The object-removal system of claim 13 wherein the second tubular sheath is made of plastic, with or without supportive wire winding or other supportive element.

15. The object-removal system of claim 9 wherein the beveled-cutting blade is symmetrically disposed around a perimeter of the distal end of the second tubular sheath.

16. The object-removal system of claim 9 wherein the snare comprises a loop or loops.

17. A method of removing an object from within a mammal comprising:
inserting an object-removal system within a mammal, the object-removal system comprising: a first tubular sheath; a second tubular sheath disposed coaxially inside the first tubular sheath wherein a distal end of the second tubular sheath comprises a beveled cutting-edge; and a snare disposed within the second tubular sheath;
attaching the snare to an object within the mammal;
moving the second tubular sheath in a longitudinal direction along the co-axis of the first and second tubular sheaths to force the beveled cutting-edge against tissue of the mammal attached to the object to cut the tissue with the beveled cutting-edge, as the second tubular sheath moves in the longitudinal direction, so that the object can be removed from the mammal; and
removing the object-removal system and the object from the mammal.

18. The method of claim 17 wherein the beveled cutting-edge has a sharpness in a range of 0.1 N to 10.0 N.

19. The method of claim 17 wherein the beveled cutting-edge comprises a blade attached to the distal end of the second tubular sheath.

20. The method of claim 17 wherein the first tubular sheath comprises a flexible material having a stiffness in a range of 0.01 to 100.0 pounds per inch.

21. The method of claim 17 wherein the second tubular sheath comprises a flexible material having a stiffness in a range of 0.01 to 100.0 pounds per inch.

22. The method of claim 17 wherein the beveled cutting-edge is made of metal or other material shaped into a cutting blade edge.

23. The method of claim 22 wherein the second tubular sheath is made of plastic, with or without supportive wire winding or other supportive element.

24. The method of claim 17 wherein the beveled-cutting edge is symmetrically disposed around a perimeter of the distal end of the second tubular sheath.

25. The method of claim 17 wherein the snare comprises a loop or loops.

26. The method of claim 17 wherein the step of attaching the snare to the object within the mammal further comprises extending the snare from the first tubular sheath to attach the snare to the object while the second tubular sheath is disposed within the first tubular sheath.

27. The method of claim 17 wherein the step of moving the second tubular sheath in the longitudinal direction along the co-axis of the first and second tubular sheaths to force the beveled cutting-edge against the tissue of the mammal attached to the object to cut the tissue with the beveled cutting-edge, as the second tubular sheath moves in the longitudinal direction, so that the object can be removed from the mammal further comprises extending the second tubular sheath from the first tubular sheath after the snare is attached to the object within the mammal.

28. The method of claim 27 further comprising retracting the second tubular sheath into the first tubular sheath after cutting the tissue with the beveled cutting-edge.

29. The method of claim 28 further comprising retracting the snare, with the object attached to the snare, within the first tubular sheath after the second tubular sheath is retracted into the first tubular sheath.

30. The method of claim 29 further comprising removing the first tubular sheath from the mammal thereby also removing the snare, the attached object, and the second tubular sheath from the mammal.

31. The method of claim 27 further comprising extending the second tubular sheath to dispose the snare and the attached objected completely within the second tubular sheath.

32. The method of claim 31 further comprising removing the second tubular sheath from the mammal, thereby also removing the snare and the attached object from the mammal, leaving the first tubular sheath disposed within the mammal.

33. The method of claim 32 further comprising removing the first tubular sheath from the mammal.

34. The method of claim 27 further comprising extending the first tubular sheath over the snare, over the attached object, and over the second tubular sheath so that each is completely disposed within the first tubular sheath.

35. The method of claim 34 further comprising removing the first tubular sheath from the mammal thereby also removing the snare, the attached object, and the first tubular sheath from the mammal.

36. The method of claim 17 wherein the object is a filter disposed within an inferior vena cava of the mammal.
